(19) United States
(12) Patent Application Publication
(10) Pub. No.: US 2004/0047853 A1
(43) Pub. Date: Mar. 11, 2004

(54) Purified proenzyme of dipeptidyl peptidase i (pro-dppi)

(76) Inventors: Soren W Dahl, Rungsted Kyst (DK); Connie Lauritzen, Rodovre (DK); John Pedersen, Niva (DK); Boris Turk, Skofljica (SI)

Correspondence Address: LAHIVE & COCKFIELD, LLP., 28 STATE STREET, BOSTON, MA, 02109, US

(21) Appl. No.: 10/297,509

(22) PCT Filed: Jun. 8, 2001
(86) PCT No.: PCT/DK01/00398

(30) Foreign Application Priority Data
Jun. 9, 2000 (DK) PA 2000 00899

(51) Int. Cl. A61K 38/48; C07H 21/04; C12N 9/64; C12P 21/02; C12N 5/06

(52) U.S. Cl. 424/94.63; 435/69.1; 435/226; 435/320.1; 435/325; 536/23.2

(57) ABSTRACT

The present invention relates to a substantially pure proenzyme of dipeptidyl peptidase I (pro-DPPI) and mutants thereof. The invention disclosed herein presents novel and fundamentally inventive means of producing substantially pure pro-DPPI in milligrams to gram scale quantities and of selectively purifying unprocessed pro-DPPI from mixtures of pro-DPPI and DPPI. The present invention further relates to biochemical and pharmaceutical applications of pro-DPPI and the generation of monoclonal and polyclonal antibodies against pro-DPPI and the uses thereof.

### FIELD OF THE INVENTION

The present invention relates to the purified proenzyme of dipeptidyl peptidase I (pro-DPPI) and to a method of purifying recombinant pro-DPPI. In addition, this invention relates to biochemical and pharmaceutical applications of pro-DPPI and to the use of pro-DPPI to generate monoclonal or polyclonal antibodies.

### BACKGROUND

Dipeptidyl peptidase I (DPPI, EC 3.4.14.1) previously known as dipeptidyl aminopeptidas I (DAPI), dipeptidyl transferase, cathepsin C and cathepsin J, is a lysosomal cysteine exo-peptidase belonging to the papain family. DPPI is widely distributed in mammalian and bird tissues and the main sources of purification of the enzyme are liver and spleen. The cDNAs encoding rat, human, murine, bovine and two Schistosoma DPPIs have been cloned and sequenced showing that the enzyme is highly conserved. The human cDNA (Paris et al. (1995) FEBS Lett. 369, 326-330) encodes a precursor (prepro-DPPI) comprising a signal peptide of 24 residues, a proregion of 206 residues, and a domain of 233 residues (“catalytic domain”). The catalytic domain contains the known catalytic residues and is 30-40% identical to the mature amino acid sequences of papain and a number of other cathepsins including cathepsins L, S, K, B and H.

The translated prepro-DPPI polypeptide is processed into the mature form by at least four chain cleavages. The signal peptide is removed during translocation of the proenzyme (pro-DPPI) into the endoplasmatic reticulum. An N-terminal proregion fragment, the residual pro-part, which is retained in the mature enzyme, is separated from the catalytic domain by excision of a minor C-terminal part of the proregion, which is named the activation peptide (FIG. 1). A heavy chain of about 164 residues and a light chain of about 69 residues are generated by cleavage of the catalytic domain. Unlike the other members of the papain family, mature DPPI consists of four subunits, each composed of the N-terminal proregion fragment, the heavy chain and the light chain. Both the residual pro-part and the heavy chain are glycosylated.

DPPI catalyses excision of dipeptides from the N-terminus of protein and peptide substrates, except if (i) the amino group of the N-terminus is blocked, (ii) the site of cleavage is on either side of a proline residue, (iii) the N-terminal residue is lysine or arginine, or (iv) the structure of the peptide or protein prevents further digestion from the N-terminus. DPPI is expressed in many tissues and is generally associated with protein degradation in the lysosomes. More recently, DPPI has been demonstrated to be capable of activating the zymogens of tryptase, chymase, cathepsin G and granzymes A, B and K in vitro. Granzymes A and B, isolated from the granules of cytotoxic lymphocytes of DPPI −/− mice, retain their activation dipeptides and are inactive, showing that DPPI is essential for granzyme activation in vivo. Furthermore, two groups (Toomes et al. (1999) Nat Genet. 23, 421-424, Hart et al. (1999) J. Med. Genet 36, 881-887) have independently identified deleterious mutations in the CTSC gene encoding human DPPI (hDPPI) as the cause of development of the autosomal recessive disorder Papillon-Lefèvre syndrome (PLS), mainly characterised by severe early onset periodontitis and palmar plantar keratosis. Together, these results suggest that DPPI is involved in a number of diseases and that this enzyme may be important in diagnosis and as a target for therapeutic intervention.

The proenzyme form of DPPI, pro-DPPI, comprises the proregion and the catalytic region of DPPI. Intermediate, glycosylated cleavage products of 31-33 kDa have previously been detected by reducing SDS-PAGE analysis following pulse-chase labelling, cell lysis and immunoprecipitation with anti-rat DPPI antibodies (bemirov et al. (1999) Biochim. Biophys. Acta 1448, 507-511). Intermediate cleavage products of 31-33 kDa have also been observed in in vitro processings of recombinant rat DPPI (Lauritzen et al. (1998) Protein Expr. Purif. 14, 434-442). Demirov et al. (Demirov et al. (1999) Biochim. Biophys. Acta 1448, 507-511) have further conducted a Percoll density fractionation experiment in transiently transfected COS 7 cells and suggest that the maturation of rat pro-DPPI is initiated In the endoplasmatic reticulum-Golgi intermediate compartment (ERGIC). A 26 kDa peptide immunoprecipitated from a light microsomal fraction, containing Golgi complex and rough endoplasmatic reticulum (RER), was recognised as the same processing intermediate and i was concluded that the processing of rat DPPI in COS 7 cells was Initiated before sorting to the lysosomes. The lower apparent mass of this intermediate, though, indicates either non-native processing of the precursor, intramolecular disulphide bonds or the detection of an unrelated peptide as a result of Insufficient antibody specificity (McGuire et al. (1992) Arch. Biochem. Biophys. 295, 280-288, Muno et al. (1993) Arch. Biochem. Biophys. 306, 103-110).

The same anti-rat DPPI antibody has been used to investigate the synthesis, transport and processing of rat DPPI in Morris hepatoma 7777 cells and in rat hepatocytes (Mainferme et al. (1985) Eur. J. Biochem. 153, 211-216). As in the other study, the DPPI proenzyme and its processing were characterised by SDS-PAGE analyses of isotope labelled and immunoprecipitated protein. Following labelling for 10 min, polypeptides with apparent masses of 59 and 63 kDa were found in Morris hepatoma 7777 cell extract whereas no protein in the cell medium was detected. Extracts prepared after 3, 24 and 72 hours of chase all contained mature DPPI with an apparent mass of 18.5 kDa. Samples of the cell medium collected at the same time points contained pro-DPPI (59 and 63 kDa) in amounts corresponding to about 30% of total DPPI whereas no mature DPPI was secreted. The secretion of pro-DPPI from freshly isolated rat hepatocytes was limited. Percoll density gradient fractionation of Morris hepatoma 7777 cell organelles after 1 hour of labelling and 0, 4 and 14 hours of chase showed that pro-DPPI was first found in the light pool containing Golgi complex and RER markers. After 4 and 14 hours of chase, pro-DPPI was localised in both the light and heavy fractions showing that pro-DPPI was also present in the lysosomes. In contrast, mature DPPI was exclusively found in the heavy Percoll density fraction containing lysosomes. These data suggest that pro-DPPI is exclusively processed within the lysosomes. The processing of pro-DPPI to mature DPPI was not affected by the inhibitor Cbz-Phe-Ala-CHN2, indicating that maturation is not dependent on lysosomal cysteine proteases.

Finally, the processing and transport of rat macrophage pro-DPPI have been studied by Muno et al. (Muno et al. (1993) Arch. Biochem. Biophys. 306, 103-110). Pro-DPPI that was detected by pulse-chase labelling, SDS-PAGE and fluorography was reported to be synthesised as a 55 kDa glycosylated polypeptide, in agreement with the calculated mass of 49,870 Da for the unglycosylated proenzyme (Ishidoh et al. (1991) J. Biol. Chem. 266, 16312-16317). Within three hours, the precursor was processed into mature enzyme. Following Percoll density fractionation, the precursor and the mature enzyme were found at low and high Percoll densities, indicating that processing had occurred within lysosomes. The presence of the residual pro-part was not reported. Both DPPI and pro-DPPI were found to be phosphorylated and the glycosylation on rat DPPI was sensitive to endoglycosidase H treatment (Muno et al. (1993) Arch. Biochem. Biophys. 306, 103-110, Demirov et al. (1999) Biochim. Biophys. Acta 1448, 507-511), indicating the presence of high mannose oligosaccharides.

In summary, the pulse-chase studies on cultured and freshly isolated rat and human cells expressing DPPI have generally shown that DPPI is expressed as a precursor. The polypeptide may be identified as a 55 kDa glycoprotein by pulse-chase labelling, lysis and immunoprecipitation, using polyclonal antibodies. Analyses of such preparations and of purified mature DPPI have shown that the precursor, pro-DPPI, oligomerises (Muno et al. (1993) Arch. Biochem. Biophys. 306, 103-1 10), becomes glycosylated at all four glycosylation sites (Cigic et al. (1998) Biochim. Biophys. Acta 1382, 143-150) and is probably transported to the lysosomes before the proteolytic maturation is initiated 0.5 to 3 hours after translation.

In all of these studies, sub-microgram amounts of impure pro-DPPI/DPPI mixtures were investigated. The polyclonal antibodies used for immunoprecipitation of pro-DPPI also precipitated labelled and unlabelled mature DPPI. Taking the rapid proenzyme activation into account, it is reasonable to assume that the mature form is more abundant In the immunoprecipitates than the proenzyme. Accordingly, analyses and uses of pro-DPPI, isolated by the described methods, will suffer from strong interference by DPPI and no method has so far been advised for its removal. Furthermore, the immunoprecipitates are formed by incubating mixtures of raw or fractionated cell extracts and antibodies at 4° C. for typically 16 hours. Clearly, a range of proteases will be present in the incubation mixture and even in the precipitates, formed in the presence of protease inhibitors, which in general do not completely block proteolysis. Therefore, processing and degradation of pro-DPPI and pro-DPPI processing intermediates can not be eliminated. In preparations of natural human DPPI from spleen and kidney, for example, 20-40% of the residual pro part chains are partially degraded (Cigic et al. (1998) Biochim. Biophys. Acta 1382, 143-150), stressing the risk of undesirable proteolysis in raw extracts.

### SUMMARY OF THE INVENTION

The present invention relates to a substantially pure proenzyme of dipeptidyl peptidase I (pro-DPPI) and mutants thereof. The invention disclosed herein presents novel and fundamentally inventive means of producing substantially pure pro-DPPI In milligram to gram scale quantities and of selectively purifying unprocessed pro-DPPI from mixtures of pro-DPPI and DPPI. In the present invention, recombinant pro-DPPI is expressed in a host cell wherein the DPPI presequence (signal sequence) directs pro-DPPI to the extracellular space/the culture medium. Pro-DPPI is then Isolated from the medium under conditions that Inhibit its processing. The present invention further relates to biochemical and pharmaceutical applications of pro-DPPI and the generation of monoclonal and polyclonal antibodies against pro-DPPI and the uses thereof.

### DETAILED DESCRIPTION

The present invention relates to a substantially pure and isolated proenzyme form of DPPI and mutants thereof. In another aspects, the present invention further relates to a method for producing and purifying a single chain polypeptide of pro-DPPI and pro-DPPI mutants from different species, such as but not limited to rat, human, dog, mouse, Schistosoma japonicum and Schistosoma mansoni. Finally, the present invention relates to biochemical and pharmaceutical applications of a single chain polypeptide corresponding to pro-DPPI and the use of said purified single chain polypeptide to generate monoclonal or polyclonal antibodies against pro-DPPI.

DPPI is widely distributed in mammalian and bird tissues. The cDNAs encoding rat, human, murine, bovine and two Schistosoma DPPIs show that the enzyme is highly conserved, as seen in FIG. 7 alignment. In one embodiment, the present invention therefore also encompasses substantially pure polypeptides that are at least 40-100%, such as 70-95%, or 70-100% identical to pro-DPPI from other mammalian species, such as rat or mouse or bovine, or even to non-mammalian species. As easily understood by a person skilled in the art, the overall % identity of a given polypeptide to a given pro-DPPI does not always accurately mirror the functional identity. Therefore, a polypeptide that shows high, such as at least 70%- 100% identity in specific conserved or functional important sites, is also comprised in the invention, although it might not show an over-all identity of at least 50%.

A preferred embodiment of the present invention thus relates to a substantially pure single chain polypeptide, which is at least 50% identical to a proenzyme of human dipeptidyl peptidase I (pro-DPPI), as shown in SEQ ID No. 1. In another embodiment of the invention, the substantially pure single chain polypeptide is at least 40% identical to human pro-DPPI, such as at least 45%, 50%, 55%, 60%, 65%, 70%, 75%, 80%, 85%, 90%, 95%, 96%, 97%, 98%, 99% or 100% identical. In an especially preferred embodiment, the substantially pure single chain polypeptide is human pro-DPPI.

The present invention further relates to a substantially pure polypeptide comprising a single chain polypeptide consisting of about 390-470 amino acid residues, said single chain polypeptide containing the internal activation peptide of pro-DPPI. In one embodiment, said internal activation peptide of pro-DPPI is at least 25% identical to the internal activation peptide of human pro-DPPI ranging from amino acid No. 134 to amino acid No. 206, as shown in SEQ ID No. 1, such as at least 25%A, 26%, 27%, 28%, 29%, 30%, 31%, 32%, 33%, 34%,35%, 40%,45%, 50%, 55%, 60%, 65%,70%, 71%, 72%, 73%,74%, 75%, 76%, 77%,78%, 79%,80%, 81%, 82%, 83%, 84% 85%, 86%, 87%, 88%, 89%, 90%, 91%, 92%, 93%, 94%, 95%; 96%, 97%, 98%, 99% or 100% identical. In one especially preferred embodiment, said internal activation peptide is the internal activation peptide of human pro-DPPI, as shown in SEQ ID No. 1.

The present invention further relates to a method for isolating a substantially pure polypeptide which is at least 40% identical to a proenzyme of human dipeptidyl peptidase I (pro-DPPI), as shown in SEQ ID No. 1, characterised by the polypeptide being isolated from a DPPI producing cell under conditions that inhibit the processing of pro-DPPI. The polypeptide can herein be either isolated directly from the cellular cytoplasm or from the extracellular space surrounding the cell, such as the medium of a cell culture.

In a preferred embodiment of the invention, a method for isolating a substantially pure polypeptide, which is at least 40% identical to a proenzyme of human dipepudyl pepfidase I (pro-DPPI), as shown in SEQ ID No. 1, comprises inserting a nucleic acid fragment containing a nucleic acid sequence encoding a polypeptide which is at least 40% Identical to human pro-DPPI into a vector, transfecting or infecting or transforming a suitable host cell or a whole organism with said vector, cultivating or growing said host cell or said host organism under suitable conditions for expressing the polypeptide, and harvesting and purifying the polypeptide under conditions that stabilise the proenzyme and/or inhibit the processing of the proenzyme. In this embodiment, the DNA fragment coding for the poly-peptide can altematively be modified by substitution, addition, insertion, deletion or rearrangement of one or more nudeotides in the fragment.

In a preferred embodiment of the invention, the method for isolating a substantially pure polypeptide which is at least 40% identical to a proenzyme of human dipeptidyl peptidase I (pro-DPPI), as shown in SEQ ID No. 1, is further characterised by initiating the isolation of pro-DPPI before any significant proteolytic processing of the proenzyme has taken place, such as maturation of the proenzyme into its mature form of at most 80% of pro-DPPI such as at most 5%, 10%, 15%, 20%, 25%, 30%, 35%, 40%, 45%, 50%, 55%, 60%, 65%, 70%, 75% or 80%. To achieve this, an especially preferred embodiment of this invention comprises isolating the expressed polypepfide not later than 4 days, such as 0.1 hour, 0.5 hour, 1 hour, 2 hours, 3 hours, 4 hours, 5 hours, 6 hours, 7 hours, 8 hours, 9 hours, 10 hours, 11hours, 12 hours, 13 hours, 14 hours, 15 hours, 16 hours, 17 hours, 18 hours, 19 hours, 20 hours, 21 hours, 22 hours, 23 hours,24 hours, 36 hours, 48 hours, 60 hours, 72 hours 84 hours or 96 hours after infection of a host cell with a vector carrying a nucleotide sequence encoding said polypeptide, or after induction of expression of said polypeptide in a host cell harbouring a vector carrying the encoding nucleotide sequence or after induction of a host cell having the nucleotide sequence encoding said polypeptide inserted in the genome.

Another preferred embodiment of the invention further comprises the introduction of conditions that stabilise the proenzyme, such as isolating the polypeptide under conditions that are characterised by having a pH value between pH 6-8, such as at critical pH values of 6, 6.2, 6.5, 6.7, 7.0, 7.2, 7.5, 7.7, or 8.0. In a most preferred embodiment, the isolation takes place under conditions that are characterised by having a pH value of 7.0.

Yet another mode of stabilising the proenzyme is encompassed in this invention by introducing conditions that will inhibit or block at least one proteolytic enzyme capable of degrading the proenzyme or converting the proenzyme into an active enzyme. Proteoiytic enzymes that will process the proenzyme are typically a variety of proteases. The invention therefore comprises Introducing one or more protease inhibitors during the expression and/or purification of the polypeptide.

The term “protease inhibitors” refers to chemical compounds, peptides and polypeptides that inhibit the activity of one or more proteases by binding covalentoy or non-covalently to the protease(s), typically at or close to the active site.

By selecting a specific inhibitor or kind of inhibitor(s), it is often possible to specifically inhibit the activity of one or more proteases or types of proteases; E-64 and cystatins (e.g. human cystatin C) are relatively non-specific covalent and non-covalent cysteine proteinase inhibitors, respectively. EDTA inhibits Ca2+ and Zn2+ dependent metalloproteases and PMSF inhibits serine proteases. In contrast, TLCK and TPCK are both inhibitors of serine and some cyst ine proteases but only TLCK inhibits trypsin and only TPCK Inhibits chymotrypsin.

The invention disclosed in the present application presents novel and fundamentally different means of producing pure pro-DPPI in milligram to gram scale quantities and of selectively purifying unprocessed pro-DPPI from mixtures of pro-DPPI and DPPI.

In one embodiment of the invention, human or rat pro-DPPI is for example expressed in an insect cell/baculovirus system. In principle, the same preproenzyme translation product and the same insect cell/baculovirus system can be used for production of mature DPPI. Importantly, the rat DPPI presequence (signal sequence), used in both constructs, which in rat targets pro-DPPI to the lysosomes, in this system surprisingly directs pro-DPPI to the extracellular space/culture medium. Clearly, this unexpected secretion of the precursor is extremely useful for preparing pro-DPPI. Normal translocation would bring the proenzyme in contact with highly potent proteases, such as cathepsins L and D, which are abundant within the lysosomes, which would inevitably result in a rapid processing (cathepsin L) or degradation (cathepsin D) of pro-DPPI.

Pro-DPPI is normally rapidly (within few hours) processed into mature, active DPPI, during translocation or more likely, within the lysosomes. Upon expression of prepro-DPPI, it is surprisingly found that pro-DPPI that escapes proteolytic maturation can be isolated from the cell medium about three days post infection and that proteolytc maturation of pro-DPPI during purification can be inhibited by using buffers with pH-values of about 7. Presumably, pro-DPPI can be isolated following intracellular expression of pro DPPI or following expression of secreted pro-DPPI N-terminally fused to other secretion signals, e.g. the human DPPI signal peptide. Pro-DPPI may also be isolated following expression of intracellular or secreted pro-DPPI in presence of one or more chemical compounds capable of inhibiting peptidases having the potential of processing and/or degrading pro-DPPI. In addition, intracellular or extracellular pro-DPPI may be expressed and isolated from other heterologous expression systems using host cells such as Saccharomyces cerevisiae, Pichia pastoris, Hansenula polymorpha, Bacillus subtilis, Streptomyces, Escherichia coli, Chinese hamster ovary cells or Drosophila cells. In addition, intracellular or extracellular pro-DPPI may be expressed and isolated from heterologous expression in higher organisms such as Insect larvae, for example larvae of silkworm.

One embodiment of the present invention comprises growing the host cell in a cell culture system, but the polypeptide can as well be expressed and isolated from a higher organism or a whole animal. The present invention therefore comprises a method wherein the host cell is a higher eukaryotic cell adapted for expression of recombinant proteins. Potential host-vector systems include but are not limited to mammalian cell systems infected with virus (e.g., vaccinia virus, adenovirus, etc.) or insect cell system infected with virus (e.g., baculovirus). The present invention also comprises a method wherein the host cell is a prokaryotic or lower eukaryotic microorganism adapted for expression of recombinant proteins. The term “prokaryotic or lower eukaryotic microorganism adapted for expression of recombinant proteins” herein refers to microorganisms including but not limited to Saccharomyces cerevisiae, Pichia pastoris, Hansenula polymorpha, Bacillus subtilis, Streptomyces or Escherichia coli. The corresponding vectors comprise vectors such as yeast vectors, plasmid DNA, bacteriophages, or cosmid DNA. The present invention also comprises a method wherein the host is a whole organism such as a whole insect, for example a larvae of silkworm, infected with a baculovirus.

In an especially preferred embodiment of the invention, a host-vector system is employed wherein the host is an insect cell such as cells derived from Trichoplusia ni or Spodoptera frugiperda and the vector is a baculovirus vector such as vectors of the type of Autographica californica multiple nuclear polyhedrosis virus or Bombyx mori nuclear polyhedrosis virus.

Mature and partially processed DPPI can be isolated from the cell medium between four to six days, preferably five days, after infection of insect cells with baculovirus. Further processing can be completely blocked with E64, showing that the processing protease is a cysteine protease, most likely the baculovirus encoded viral cathepsin protease v-cath, which is homologous to cathepsin L. The v-cath protease is either secreted into the cell medium or released from the insect cells upon baculovirus induced cell lysis. Cell lysis is significant on day four and five after infection in accordance with observations that pro-DPPI is preferentially isolated from the medium 3 days post infection. The present invention therefore further relates to isolating and purifying said polypeptide from insect cells at the latest 96 hours (4 days) post infection such as 24, 36, 48, 60, 72 or 84 hours post infection.

The method for purifying a substantially pure proenzyme of dipeptidyl peptidase I (pro-DPPI) comprised in the present invention is typically characterised by yielding an outcome of 1-500 mg, such as 10-100 mg, 10-50 mg, 10-25 mg, or 1-15 mg of substantially pure protein purified per litre of cell culture.

When the method is conducted using an insect cell culture, the method is typically characterised by yielding an outcome of 10-25 mg per litre of culture or per 1-8×10⁹ cells.

In the present invention, extracellular pro-DPPI is isolated by ammonium sulphate fractionation, hydrophobic interaction chromatography, desalting and subtractive anion exchange. Other chromatographic and fractionation principles may also be used in purification of pro-DPPI, e.g. purification by anion exchange, cation exchange, high performance liquid chromatography (HPLC), immobilised metal affinity chromatography (IMAC), affinity chromatography or precipitation. Hereby new means of protein purification are generated that allow pro-DPPI to be selectively isolated from mature DPPI, said means being characterised by employing ammonium sulphate fractioning, hydrophobic interaction, desalting, and anion exchange chromatography. As exemplified in example 8, an especially preferred embodiment of the present invention relates to such a means further characterised by employing an anion exchange matrix which selectively binds mature DPPI.

The important regulatory functions of DPPI, described in the introduction, makes investigations of the activation of DPPI itself of significant interest. Peptidases capable of converting pro-DPPI into active DPPI and possible co-factors, e.g. one or more glycosaminoglycans and metal ions, can be identified by reacting pro-DPPI with said peptidases and co-factors under various conditions, e.g. conditions that are similar to those experienced by pro-DPPI and DPPI in vivo. The activation of recombinant immature rat DPPI in vitro preferably occurs at pH 4.5 (Lauritzen et al. (1998) Protein Expr. Purif. 14, 434-442). Candidate peptidases are pro-DPPI itself, active DPPI, lysosomal cysteine peptidases, peptidases other than lysosomal cysteine peptidases and any combination hereof. Most peptidases in the papain family, including papain and cathepsins L, B, S and K are capable of autocatalytic activation whereas the cysteine exopeptidase cathepsin X is not The properties of pro-DPPI have so far not been characterised and K is not known if this proenzyme can autoactivate through intramolecular cleavages, by interacting with another pro-DPPI proenzyme, or with active DPPI, or through any combination of these possibilities. In any situation, pro-DPPI activation requires endoproteolytic activity of at I ast one functional component and different groups have reported DPPI endopeptidase activity.

The processing of pro-DPPI in vivo has been reported to be initiated before translocation to the lysosomes (Demirov et al. (1999) Biochim. Biophys. Acta 1448, 507-511) and to be independent of cysteine peptidase activity (Mainferme et al. (1985) Eur. J. Biochem. 153, 211-216) generally associated with the lysosomes or to take place within the lysosomes (Mainferme et al. (1985) Eur. J. Biochem. 153, 211-216, Muno et al. (1993) Arch. Biochem. Biophys. 306, 103-110). Clearly, the availability of pure pro-DPPI is a valuable tool in investigating the basic principles of pro-DPPI maturation. The reaction can be either autocatalytic or (primarily) non-autocatalytic and in the latter case, pro-DPPI can be reacted with cysteine and non-cysteine peptidases to determine if enzymes from one or more peptidase classes are required for maturation and processing. In particular, the interaction of pro-DPPI with cysteine peptidases, which are abundant in the lysosomes, and other lysosomal peptidases will give a strong indication as to the subcellular compartment(s) wherein pro-DPPI processing occurs in vivo.

The present invention therefore comprises the use of a substantially pure polypeptide as defined above for the identification of proteases that are capable of activating DPPI, by reacting said pro-DPPI with proteases of choice. Said activation of DPPI may optionally be monitored by e.g. DPPI assay and SDS-PAGE analysis.

Purified recombinant human pro-DPPI and the processed and active mature form have been characterised in detail and compared with natural human DPPI. With respect to the primary, secondary and quaternary structures, the recombinant enzyme forms are highly similar to their natural counterparts.

Furthermore, detailed biochemical, biophysical and enzymatic analysis of the prepared proenzyme show that the material is of the highest quality.

As compared to natural DPPI and pro-DPPI, the recombinant products have very similar folds and glycosylation patterns and the same oligomeric structures. Furthermore, recombinant pro-DPPI can be activated 2,000-fold by addition of papain, showing that mature DPPI can be efficiently removed from the proenzyme preparations by subtractive anion exchange at pH 7.0 and that pro-DPPI is a true and potentially active precursor of DPPI. This allows for Identification of natural pro-DPPI binding proteins. Meaning that novel functions of DPPI and pro-DPPI may be identified if proteins that specifically bind to pro-DPPI can be identified.

Due to the superior quality of the purified polypeptide according to the invention, the peptide bonds in pro-DPPI, which are cleaved by the peptidases capable of converting pro-DPPI to active DPPI, and the order(s) of these bond cleavages can be determined. To do this, pro-DPPI is treated with one of these peptidases under the desired conditions and in presence or absence of one or more co-factors. In these in vitro studies, activation of DPPI can be analysed by measuring the DPPI activity and the proteolytic processing of pro-DPPI can be visualised by SDS-PAGE analysis of samples collected before, during and after processing. Persons skilled in the art of biochemical and biophysical protein analysis can further investigate selected peptides identified by gel electrophoresis, e.g. by mass spectrometry and amino acid sequencing following electroblotting onto a polyvinylidene difluoride (PVDF) membrane.

The present invention subsequently relates to the use of a substantially pure polypeptide as defined above for the identification of natural pro-DPPI binding proteins and for the identification of pro-DPPI processing pathways in vivo and In vitro, by isolating and characterising intermediate pro-DPPI processing products.

The substantially pure polypeptide of the present invention will be of use as a medicament. For example: pro-DPPI can be used for developing vaccines against pro-DPPI by a method described in EP752886B1. This patent describes a method for the modification of a protein so as to induce antibody response to the unmodified protein followed by administration of the modified protein to the host. Pro-DPPI modified In the described way can be used as an autovaccine against pro-DPPI and/or DPPI.

An autovaccine based on modified forms of pro-DPPI may be a vaccine against pro-DPPI and/or DPPI mediated conditions or diseases including but not limited to mast cell, macrophages, neutrophils, and cytotoxic T lymphocytes related conditions and/or diseases, such as asthma, psoriasis, inflammatory bowel disease, graft-versus-host-disease, cardiac hypertrophy, heart failure, atherosclerorosis, perionditis, rheumatoid arthritis, allergic rhinitis, arteriosclerosis, organ allograft rejection, multiple sclerosis and myasthenia grais.

Another aspect of the invention Is the production and use of pro-DPPI antibodies. Pro-DPPI differs from mature DPPI by its primary and also by the secondary and tertiary structure making it possible to produce antibodies specific for this form of the enzyme. The term “antibody” is used in the broadest sense and specifically covers monoclonal antibodies, polyclonal antibodies, and also biologically active fragments of antibodies, preferably fragments containing the antigen binding regions.

Purified pro-DPPI can be used to immunise animals in order to raise polyclonal antibodies by conventional methods known in the art (see, for instance, Antibodies: A laboratory manual, Harlow and Lane, Cold Spring Harbor Laboratory Press, 1988). Polydonal antibodies are preferably raised in a mammal, for example, rat, mouse, rabbit or higher mammal, for example, primate, by multiple injections of the antigen. It may be useful to conjugate the antigen to a protein that is immunogenic in the species to be immunised. Animals are immunised with the antigen, immunogenic conjugates, or derivatives by injecting the solution intradermally at multiple sites. Later, the animals are boosted with the antigen or conjugate until the titre plateau, the animals are bled and the polyclonal antibodies are purified from serum.

The term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies, directed against different determinants (epitopes), each monoclonal antibody is directed against a single determinant on the antigen. Monoclonal antibodies can be prepared by a number of methods well known to those skilled in the art For example, monoclonal antibodies to be used in accordance with the present invention may be made by the hybridoma method first described by Kohler and Milstein (Nature (1975) 256,495-497) or may be made by recombinant DNA methods (see, e.g., U.S. Pat. No. 4,816,567). Monoclonal antibodies may also be isolated from phage antibody libraries using the techniques described by Clackson et al. (Nature (1991) 352,624628) and Marks et al. (J. Mol. Biol. (1991) 222, 581-597).

The present invention comprises a substantially pure polypeptide as defined above for the manufacture of a diagnostic agent such as a monoclonal or polyclonal antibody which binds to pro-DPPI or partially processed pro-DPPI, for the production of pro-DPPI immunogenic substances or an immunologic composition comprising a substantially pure polypeptide. The immunologic composition can further comprise an immunologically and pharmaceutically acceptable carrier, vehicle or adjuvant.

Polyclonal or, preferably, monoclonal antibodies have a number of useful properties. For example, they can be used as specific immunoprecipitation reagents to detect the presence of the antigen. This is done by coupling the antigen-antibody reaction to suitable detection techniques, such as labelling the purified antibodies with iodine, fluorochromes, biotin, radioisotopes or with enzymes that are capable of catalysing a desired reaction.

In one embodiment of the present invention, a pro-DPPI antibody is thus provided with,a detectable label. The label can be selected from the group consisting of enzymes, fluorophores, radioactive isotopes, gold particles and complexing agents such as biotin. Further comprised in the scope of the present invention is a method for detecting vertebrate and parasite DPPI-gene products in tissue comprising treating the tissue with a specific antibody, which is developed using pure unprocessed or partially processed pro-DPPI and which is capable of binding to said gene product, for a time sufficient to allow a complex to form between said antibody and any pro-DPPI present in the tissue, and visualising the presence of the complex, if any. Also, a method for quantifying the amount of vertebrate and parasite DPPI-gene products In biological material is comprised in the invention, comprising binding a specific monoclonal or polydonal antibody, developed using pure unprocessed or partially processed pro-DPPI, to said pro-DPPI and detecting the presence of bound antibodies or assessing the amount of bound antibodies.

A wide variety of techniques and protocols exist for detecting an antigen In a sample suspected of containing the antigen, e.g. radio immune assay (RIA), enzyme multiplied immunoassay technique (EMIT), or enzyme-linked immunosorbent assay (ELISA). For reviews, see e.g. Self (1996) Curr. Opin. Biotechnol. 7, 60-65 and Ronald and Stimson (1998) Parasitology 117, Suppl. 13-27.

The present invention thus further includes a method wherein the assessment of the amount of bound antibodies is performed by a method of the ELISA type or wherein the assessment of the amount of bound antibodies is performed by a radioimmune assay.

Antibodies are thus the foundation of immunodiagnostic tests for many antigenic substances. Pro-DPPI specific antibodies may have use in diagnosing conditions or diseases wherein pro-DPPI or DPPI are involved. They can be employed using tissues or lysates of tissues, or in detecting and/or quantifying subject epitopes in blood or serum.

Also, pro-DPPI specific antibodies can be used in studies of the activation pathway or pathways for DPPI in vivo, e.g. by immunofluorescence microscopy or immunohistochemical staining. As a component in immunoassays, pro-DPPI antigen can be detected, localised and/or quantified both within the single cells and within tissues. In one embodiment of the invention, the biological material is a tissue sample, and the method of detecting pro-DPPI within the material is used for the diagnosis of and/or assessment of prognosis in conditions and/or diseases, such as asthma, psoriasis, inflammatory bowel disease, graft-versus-host-disease, cardiac hypertrophy, heart failure, atheroselerorosis, perionditis, rheumatoid arthritis, allergic rhinitis, arteriosclerosis, organ allograft rejection, multiple sclerosis and myasthenia grais and conditions and/or diseases related to excessive or reduced apoptosis.

In a study made by Mainferme et al. (Eur. J. Biochem. (1985) 153, 211-216) using Morris hepatoma 7777 cells in culture, it was found that up to 30% of newly produced DPPI was secreted to the medium as pro-DPPI. It is likely that some types of cells, for example mast cells which is one of the major identifiable sources of DPPI, also secrete pro-DPPI in vivo, and that this enzyme is first proteolytically activated under certain conditions. High-affinity pro-DPPI specific antibodies may be directly injected into subjects suffering from conditions or diseases associated with elevated levels of extracellular DPPI or pro-DPPI. Whole body diagnosis and therapeutic treatment is made possible because injected antibodies are directed to specific target disease tissues, and thus can be used either to determine the presence of the disease by carrying with them a suitable label, or to attack the diseased tissue by carrying a suitable drug. Further, the high-affinity pro-DPPI specific antibodies may prevent proteolytic cleavage and activation of secreted pro-DPPI, and may be used as drugs.

A major impediment for using e.g. murine monoclonal pro-DPPI antibodies for diagnosis or therapy in humans is the potential Immunogenicty. Therefore, humanised forms of non-human antibodies may be prepared for this purpose. Humanised antibodies contain minimal sequence derived from non-human immunoglobulin. For the most part, humanised antibodies are human immunoglobulins (recipient antibody) wherein hypervariable region residues of the recipient are replaced by hypervariable region residues from a non-human species (donor antibody) such as mouse, rat, rabbit or non-human primate having the desired specificity, affinity, and capacity. For reviews, see e.g. Presta (1992) Curr. Op. Struct. Biol. 2, 593-596 and Jollife (1993) Int. Rev. Immunol. 10, 241-250.

Pure pro-DPPI can be used for preparing a pharmaceutical preparation containing pro-DPPI for treatment of a specific and localised need of pro-DPPI and/or active DPPI. These useful areas include but are not limited to the treatment of prepathologic conditions and chronic or acute diseases, which are characterised by differences in the amount or distribution of pro-DPPI and/or active DPPI. A pharmaceutical composition will comprise a substantially pure polypeptide as defined above and will be formulated with pharmaceutically acceptable stabilisers including salts, detergents and polymers, such as sodium chloride, sodium phosphate, polysorbat, and polyethyleneglycol, and/or formulated with pharmaceutically acceptable adjuvants such as saponin, quil A and calcium phosphate.

Alternatively, substantially pure pro-DPPI can indirectly be used to Inhibit the processing of pro-DPPI, by administration of a substance that binds to pro-DPPI and thereby inhibiting the processing of pro-DPPI into mature DPPI, and thereby inhibiting the localised catalytic activity of DPPI. Thus, the present invention further comprises the use of an inhibitor substance for the processing of pro-DPPI into mature DPPI as a medicament as well as the use of an inhibitor substance for the processing of pro-DPPI into mature DPPI for the preparation of a medicament for the treatment of conditions and/or diseases, such as asthma, psoriasis, Inflammatory bowel disease, graft-versus-host-disease, cardiac hypertrophy, heart failure, atheroscierorosis, perionditis, rheumatoid arthritis, allergic rhinitis, arteriosclerosis, organ allograft rejection, multiple sclerosis and myasthenia grais and conditions and/or diseases related to excessive or reduced apoptosis.

A preferred embodiment of this method is one wherein the substance is a monoclonal antibody that is capable of binding to said polypeptide. Said monoclonal antibody can be used for treating conditions and/or diseases, such as asthma, psoriasis, inflammatory bowel disease, graft-versus-host-disease, cardiac hypertrophy, heart failure, atherosclerorosis, perionditis, rheumatoid arthritis, allergic rhinitis, arteriosclerosis, organ allograft rejection, multiple sclerosis and myasthenia grais and conditions and/or diseases related to excessive or reduced apoptosis in a mammal, by inhibiting the processing of pro-DPPI, and thereby inhibiting the localised catalytic activity of DPPI. In a most preferred embodiment of the invention, said mammal is a human.

It has been shown that mast cells are the major source of DPPI in airways and alveolar macrophages in lung. It has also been show that DPPI is stored in mast-cell secretory granules. DPPI stored in these secretory granules is subject to regulated release outside the cell where it can interact with potential extracellular targets and studies have also reported the secretion of DPPI by cultured mast cells. Evidence supporting the packaging of DPPI from mast-cell secretory granules is the finding of DPPI activity in the granular fraction of cell extracts and its co-release with tryptase, a known secretory granule-associated protease. Furthermore, finding of DPPI immunoreactivity in granules of cultured mast cells provides further evidence that DPPI is stored in mast-cell secretory granules. From these granules, DPPI may then be released following mast cell activation by mediators such as substance P or IgE-bound antigen, which cross-links the IgE receptor of a degranulating mast cell.

Secretion of DPPI from cells is not limited to mast cells. Cytotoxic T lymphocytes, neutrophils, and macrophages also secrete DPPI. For example, cytotoxic T lymphocytes stimulated with ionomycin secretes DPPI. The possibility of neutrophil secretion of DPPI is supported by observations that DPPI and elastase activities co-localise to the granular fraction of myelomono-cytic cells and that elastase is secreted from activated neutrophils. Macrophage secretion of DPPI is also indirectly supported by the observation that macrophages secrete cathepsins B, L, and S, three lysosomal cysteine proteases related to DPPI. Further, DPPI has been shown to be secreted by rat peritoneal macrophages treated with an anti-mannose 6-phosphate receptor antibody. Thus, DPPI is likely to be secreted by most of the major cell types known to synthesise it, suggesting that its extracellular activities are not limited to mast cells. The fact that DPPI is secreted by a number of specific cell types with specific localisation in the body indicates a specific function for DPPI -at the site(s) of its secretion. After secretion, DPPI may cleave proteins and peptides in the extra-cellular space. In doing so, DPPI may influence cellular functions or matrix remodelling. These effects would probably be limited to the local region around the DPPI secreting cell because circulating proteases Inhibitors such as the cysteine protease inhibitors (cystafins), should Inhibit DPPI as it diffuses away from the cell.

Targets for DPPI can include matrix proteins as well as small bioactive peptides or other proteins such as enzymes and cytokines. DPPI may participate In matrix remodelling by secondarily removing amino-terminal dipeptides from matrix proteins that have been cleaved endo-proteolytically by other proteases, thereby solubilising the degraded matrix components and facilitating their tissue clearance. One identified intracellular role for DPPI is activation of proforms of the serine proteases tryptase, chymase, cathepsin G, elastase and granzymes A, B and K. A possible role for secreted DPPI may also be an. extracellular activation of the secreted proforms of these proteases. DPPI may also participate in degradation of biological active peptides including but not limited to glucagon, glucagon-like protein I and II (GLP-I and GLP-II), secretin, vasoactive intestinal peptide, gastric inhibitory peptide and angiotensin. The high levels of DPPI expression in alveolar macrophages also suggest that the enzyme plays a role in macrophage, functions. One possibility is participation in antigen presentation. Cysteine proteases such as cathepsins L and S degrade the invariant chain, an essential process for antigen presentation by major histocompatibility complex (MHC) class II proteins. In addition, the cysteine proteases legumaine and cathepsin B cleave antigens into fragments for presentation. The optimal length of an antigen for MHC class II presentation is 15 to 22 amino acids. One additional possible role for DPPI in antigen presentation is to process antigens into fragments suitable for presentation by MHC. For the described conditions and other related situations where DPPI may have a physiological role related to the amount or distribution of pro-DPPI and/or active DPPI, pro-DPPI will be very useful In regulating the amount of active DPPI at (a) specific site(s). Because circulating protease inhibitors such as cystatins should inhibit DPPI if DPPI Is injected into the body, active DPPI will not be useful for treating condition where DPPI is needed for a given role at a specific localisation in the body. However, as shown in example 12 cathepsins L and S are activators of DPPI. Macrophages and other cells secreting DPPI also s crete thes two lysosomal DPPI-activating cysteine proteases. Pro-DPPI administered to the body can therefore be activated to DPPI at the specific localisation in the body housing the cells secreting pro-DPPI and/or cells secreting any DPPI-activating enzyme(s) including but not limited to cathepsins L and S.

The useful areas where pro-DPPI can be used for treatment of prepathologic-condition and chronic or acute diseases include but are not limited to the treatment of conditions involving cells secreting DPPI. These conditions and diseases includes but are not limited to mast cell, macrophages, neutrophils, and cytotoxic T lymphocytes related conditions and/or diseases, such as asthma, psoriasis, inflammatory bowel disease, graft-versus-host-disease, cardiac hypertrophy, heart failure, atherosclerorosis, perionditis, rheumatoid arthritis, allergic rhinitis, arteriosclerosis, organ allograft rejection, multiple sclerosis and myasthenia grais.

Using pro-DPPI prepared by the disclosed method, chemical compounds that bind to the proenzyme can for the first time be identified, e.g. in biochemical or cell-based high throughput screening experiments known to persons skilled in the art of drug screening. Compounds, which stabilise or destabilise pro-DPPI could be desirable as pharmaceutical agents in any body fluid, including blood, serum, cerebrospinal fluid and lymph, to e.g. stabilise pro-DPPI as an inactive precursor or to promote the activation or clearance of pro-DPPI. In other words, compounds that interfere with the pro-DPPI activation or clearance rates are potential pharmaceuticals for regulating the levels of DPPI activity.

Definitions

Substantially pure is herein used to describe a polypeptide that is at least 70% pure, such as 75%, 80%, 85%, 90%, 91%, 92%, 93%, 94%, 95%, 96%, 97%, 98%, 99%, 99.5% or such as 100% pure from other polypeptide components. The % value herein indicates % (w/w).

In descriptions of homology between amino acid sequences, the term “identical” refers to amino acid residues of the same kind that are matched following pairwise Clustal W 1.8 alignment (Thompson et al. (1994) Nucleic Acids Res. 22, 4673-4680) of two known polypeptide sequences. The percentage of amino acid sequence identity between such two known polypeptide sequences is determined as the percentage of matched residues that are identical relative to the total number of matched residues.

Identity as known in the art, is a relationship between two or more polypeptide sequences or two or more polynucdeotide sequences, as determined by comparing the sequences. In the art, “identity” also means the degree of sequence relatedness between polypeptide or polynucleotide sequences, as the case may be, as determined by the matchbetween strings of such sequences. “Identity” and “Similarity” can readily be calculated by known methods.

By a polypeptide having an amino acid sequence at least, for example, 95% identical to a reference amino acid sequence as shown in SEQ ID No. 1, corresponding to human pro-DPPI polypeptide, is intended that the amino acid sequence of the polypeptide is identical to the reference sequence except that the amino acid sequence may include up to 5 point mutations per each 100 amino acids of the reference amino acid sequence as shown in SEQ ID No. 1, corresponding to human pro-DPPI polypeptide. In other words, to obtain a polypeptide having an amino acid sequence at least 95% identical to a reference amino acid sequence: up to 5% of the amino acids in the reference sequence may be deleted or substituted or added to with another amino add, or a number of amino acids up to 5% of the total amino acids in the reference sequence may be inserted into the reference sequence. These mutations of the reference sequence may occur at the amino or carboxy terminal positions of the reference amino acid sequence or anywhere between those terminal positions, interspersed either individually among amino acids in the reference sequence or in one or more contiguous groups within the reference sequence.

The preferred computer program to determine the level of sequence identity between two amino acid sequence is Clustal W 1.8 (Thompson et al. (1994) Nucleic Acids Res. 22, 4673-4680).

The term “pro-DPPI” refers to the single chain proenzyme form of dipeptidyl peptidase I also known as DPPI, DAPI, dipeptidyl aminopepudase I, cathepsin C, cathepsin J, dipeptidyl transferase, dipeptidyl arylamidase and glucagon degrading enzyme. The term also refers to any polypeptide which shares at least 40% amino acid sequence identity to the amino acid sequence of human pro-DPPI (FIG. 1) and at least 50% amino acid sequence identity to the catalytic domain of human DPPI as determined by pair-wise sequence alignment using the computer program Clustal W 1.8 (Thompson et al. (1994) Nucleic Acids Res. 22, 4673-4680). The enzyme may be of mammalian or insect origin. Alternatively, the enzymes may b obtained by expressing the genes or cDNAs encoding the enzymes or enzyme mutants or enzyme fusions or hybrids hereof in a recombinant system.

The term “DPPI” generally refers to a mature form of DPPI. In other uses of the term, “DPPI” refers to the enzyme in general, irrespective of its processing state, e.g. unprocessed, partially processed or fully processed, or partially or fully degraded. As an example, this is indicated when “DPPI” is expressed in insect cells. The term “vector” stands for a nucleic acid compound used for the transformation of cells. A vector contains a polynucleotide sequence corresponding to appropriate protein molecules which, when combined with appropriate control sequences, confer specific properties on the host cell to be transformed. Plasmids, viruses and bacteriophage are suitable vectors. Artificial vectors can be constructed by cutting and joining DNA molecules from different sources using restriction enzymes and ligases. The term vector also includes recombinant DNA cloning vectors and recombinant DNA expression vectors.

In the present context the term signal sequence means an amino acid sequence found at the amino-terminal end of a secretory protein that guides the protein to the Endoplasmatic Reticulum membrane, where it is usually enzymatically removed. The role of said signal sequence is properly to initiate entry through the barrier of a lipid bilayer.

The term “processed” refers to a molecule that has been subjected to a modification, changing it from one form to another. More specifically, the term “processed” refers to a form of pro-DPPI which has been subjected to at least one post-translational chain cleavage (per subunit) in addition to any cleavage resulting in the excision of a signal peptide.

The term “mature” refers to pro-DPPI following native like processing. The mature product, DPPI, contains at least about 80% of the residual pro-part, 90% of the heavy and light chain residues and less than 10% of the activation peptide residues.

The term “heavy chain” refers to the major peptide in the catalytic domain of DPPI. In human DPPI, the heavy chain constitutes the proenzyme residues 200-370 or more specifically residues 204-370 or residues 206-370 or even more specifically residues 207-370.

The term “light chain” refers to the minor peptide in the catalytic domain of DPPI. In human DPPI, the light chain constitutes the proenzyme residues 371-439.

The term “proregion” refers to the region N-terminal of the catalytic domain region of pro-DPPI. In human pro-DPPI, the proregion constitutes residues 1-206 or residues 1-205 or residues 1-203 or residues 1-199.

The term “activation peptide” refers to the part of the proregion in pro-DPPI, which is excised in the mature form of the enzyme. In human DPPI, the activation peptide constitutes residues 120-206 but may also constitute residues 120-199, 120-203, 120-205 or 120-206 or residues 134-199, 134-203, 134-205 or 134-206. The N-terminal and C-terminal residues are not confirmed and may vary. The activation peptide of pro-DPPI is thought to be homologous to the propeptides of cathepsins L and S.

The term “residual pro-part” refers to the part of the proregion in pro-DPPI, which is not excised In the mature form of the enzyme. In human DPPI, the residual pro-part constitutes residues 1-120 or 1-133. The C-terminal residue is not confirmed and may vary.

The term “catalytic domain” refers to the structural unit, which is formed by the heavy chain and light chain in mature DPPI. The structure of the catalytic domain is presumed to be homologous to the structures of mature papain and cathepsins L, S, B etc.

The term “inhibitor” refers to a compound that has the potential of decreasing the activity of DPPI by Interacting with the enzyme.

The term “mutant” refers to a polypeptide, which is obtained by replacing or adding or deleting at least one amino acid residue in native pro-DPPI with a different amino acid residue. Mutation can be accomplished by adding and/or deleting one or more residues in any position of the polypeptide corresponding to pro-DPPI.

The term “homologue” refers to any polypeptide, which shares at least 25% amino acid sequence identity to the reference protein as determined by pair-wise sequence alignment using the computer program Clustal W 1.8. (Thompson et al. (1994) Nucleic Acids Res. 22, 4673-4680).

The term “subunit” refers to a part of DPPI, which originates from the same precursor peptide or modified precursor peptide. Native DPPI consists of four subunits formed by association of four modified translation products.

The term “preparative scale” refers to expression and/or isolation of a protein in an amount larger than 0.1 mg.

The term “active site” refers to the cavity in each DPPI subunit into which the substrate binds and wherein the catalytic residues are located.

The term “catalytic residues” refers to the cysteine and histidine residues in each DPPI subunit, which participate in the catalytic reaction. In human pro-DPPI, the catalytic residues are cysteine 234 and histidine 381.

When used to describe a preparation of a protein, the term “pure” refers to a preparation wherein at least 80% (w/w) of all protein material in said preparation is said protein.

### FIGURE L GENDS

FIG. 1.

Displays the amino acid sequence of human pro-DPPI. The residues in the residual pro part, the activation peptide and in the heavy and light chains are indicated and the catalytic and glycosylated residues are marked with asterisks and hexagons, respectively.

FIG. 2

Displays SDS-PAGE analysis of recombinant human DPPI after deglycosylation of wild type and single site N-glycosylation mutants.

FIG. 3

Displays far-(left panel) and near-(right panel) UV CD spectra of native human DPPI (thin lines), recombinant human DPPI (thick lines) and recombinant human pro-DPPI (broken lines).

FIG. 4

Displays SDS-PAGE analysis of human pro-DPPI following treatment with human cathepsins B, S and L.

FIG. 5

Displays appearance of DPPI activity following activation of pro-hDPPI by human cathepsins B, S and L

FIG. 6

Displays the purity and stability of different fractions during the purification of human pro DPPI.

FIG. 7

Displays a CLUSTAL W (1.81) Multiple Sequence Alignment of the polypeptide sequences of rat, human, dog, mouse, Schistosoma japonicum and Schistosoma mansoni pro-DPPI's. Scoring matrix blosum. Opening gap penalty:10. Gap separation penalty: 1.

### EXAMPLES

### Example 1

Construction of Transfer Vector for Rat Prepro-DPPI

The construction of a baculovirus transfer vector termed pCLU10-4 (identical to the vector termed pVL1393-DPPI) encoding rat DPPI preproenzyme is described in (Lauritzen et al. (1998) Protein Expr. Purif. 14, 434-442). Here, rat cDNA was prepared based on the sequence published by Ishidoh et al. (J. Biol. Chem. (1991) 266, 16312-16317). The rat prepro-DPPI encoding region was amplified by polymerase chain reaction (PCR) from the cDNA pool to generate restriction sites at the 5′ and 3′ ends of the portion of the sequence coding for the residues Met (−24)-Letu (438). Two oligonucleotide primers, 5′-GCT CTC CGG GCG CCG TCA ACC and 5′-GCT CTA GAT CTU ACA ATT TAG GAA TCG GTA TGG C (no. 6343 and no. 7436 from DNA Technology, Aahus, Denmark) were designed to specifically amplify the DNA sequence as well as to incorporate a HindII restriction site at the 5′ end and a BglII restriction site and a TAA stop codon at the 3′ end of the coding sequence.

PCR amplification was performed with these two oligonucleotide primers for 30 complete PCR cycles with each cycle involving a 1 minute denaturation step at 95° C., a 1 minute annealing step at 65° C., and a 1.5 minute polymerisation step at 72° C. The cycles were followed by an extension step of 10 minutes at 72° C.

The 1395 bp fragment obtained from PCR amplification and digestion with HindII and BglII was ligated into baculovirus transfer vector pVL1393 (Catalogue #21201 P, Pharmingen, San Diego, Calif.) at the SmaI and BglII cloning site within a multiple cloning site. The resulting transfer vector CLU10-4 also carries a strong baculovirus polyhedrin promoter, a flanking polyhedrin region from the AcNPV virus as well as an E. coli origin of replication and an ampicillin resistance gene for plasmid amplification and selection in E. coli. As cloned on pCLU10-4, the fragment encoding rat DPPI is expressed under the control of the polyhedrin promoter as prepro-DPPI i.e. with the endogenous signal sequence serving to direct secretion of rat DPPI into the culture medium. Proper vector construction was confirmed by nucleotide sequencing of the coding region on the constructed plasmid.

### Example 2

Construction of Transfer Vector for Human Prepro-DPPI

A transfer vector termed pCLU70-1 encoding human DPPI proenzyme N-terminally fused to the signal sequence (pre-sequence) of rat DPPI preproenzyme was prepared as follows. The human pro-DPPI cDNA, previously described as a 1.9 kb full length prepro-hDPPI construct in pGEM-11Zf(−) (Paris et al. (1995) FEBS Lett. 369, 326-330) was amplified by polymerase chain reaction (PCR) to generate restriction sites at the 5′ and 3′ ends, respectively, of the portion of the hDPPI sequence coding for pro-DPPI residues-2-439 lacking all but the two N-terminal residues of the endogenous signal peptide and starting with Ser (−2) and ending with Leu (439). Two oligonucleotide primers, 5′-AAA CTG TGA GCT CCG ACA CAC CTG CCA ACT GCA-3′ (NT-HSCATC from TAGCopenhagen, Copenhagen, Denmark) and 5′-ACT GAT GCA GAT CTT TAT GAA ATA CTG GAA GGC-3′ (HS-RBGL from Gibco BRL, Life Technologies, Gaithersburg, Md.), were designed to specifically amplify the DNA sequence as well as incorporating a SacI restriction site at the 5′ end and maintaining a TAG stop codon and creating a BglII restriction site at the 3′ end of the coding sequence.

PCR amplification was performed with these two oligonucleotide primers for 25 complete PCR cycles with each cycle involving a 1 minute denaturation step at 95° C., a 1 minute annealing step at 62° C., and a 1 minute polymerisation step at 72° C. The cycles were followed by an extension step of 10 minutes at 72° C.

The fragment amplified from human DPPI cDNA and digested with SacI and BglII was ligated into the baculovirus transfer vector pCLU10-4 (described in Example 1) at the SacI and BglII sites. Thereby, the rat pro-DPPI sequence (coding the residues (−)2-438) was deleted and replaced by the human sequence. As cloned on the resulting vector pCLU70-1, the gene fragment is expressed as a fusion between the residues 1-439 of the hDPPI sequence and the entire signal sequence for the rat DPPI protein serving to direct secretion of human DPPI Into the culture medium. Proper vector construction was confirmed by nucleotide sequencing of the entire prepro-DPPI coding region on the constructed plasmid.

### Example 3

Preparation of Recombinant Baculoviruses

For the preparation of recombinant baculoviral stocks, pCLU10-4 and pCLU70-1 were transformed into E. coli strain TOP10 (Catalogue #C4040-10, Invitrogen, Groningen, The Netherlands), amplified and purified by well-established methods (Wizard Plus SV Minipreps DNA Purification Systems, Promega, Madison, Wis.). The purified transfer vectors pCLU10-4 and pCLU70-1 were co-transfected with BaculoGold DNA (Catalogue #21 100D, Pharmingen, San Diego, Calif.) into Spodoptera frugiperda Sf9 cells (American Type Culture Collection, Rockville, Md.) using the calcium phosphate protocol (Gruenwald et al. (1993) Procedures and Methods Manual, 2nd ed., Pharmigen, San Diego, Calif. p.44-49). BaculoGold is a modified baculovirus DNA that contains a lethal deletion and accordingly cannot encode for a viable virus by itself. When co-transfected with a complementing transfer plasmid, such as pCLU10-4 or pCLU70-1, carrying the essential gene lacking in BaculoGold, the lethal deletion is rescued and viable virus particles can be reconstituted inside transfected insect cells.

Sf9 cells were maintained and propagated at 27-28° C. as 50-mi suspension cultures in roller bottles and seeded as monolayers when used for co-transfection, plaque assays or small-scale amplifications. Sf9 cells were for all purposes grown in BaculoGold Serum-Free medium (Catalogue #21228M, 17 Pharmigen, San Diego, Calif.) supplemented with 5% heat inactivated foetal bovine serum (Gibco BRL, Catalogue #10108-157). Gentamycin (Gibco BRL, Catalogue # 15750-037) to 50 mg/ml were added to cultures used for co-transfection and plaque assays.

### Example 4

Virus Purification, Verification, and Amplification

The virus generated in the cotransfection with BaculoGold DNA and transfer vectors were plaque purified (Gruenwald et al. (1993) Procedures and Methods Manual, 2nd ed., Pharmigen, San Diego, Calif. p. 51-52) to generate virus particles for further infections. The structure of the purified viruses was verified by PCRF Picked plaques were suspended in 100 μl medium and Incubated at 4° C. for >18 hours. 15 μl of this suspension were used to infect High Five TM (Trichoplusia insect cells) (BTI-TN-5B1-4) (Invitrogen) In monolayers. High Five TM cells were maintained and propagated at 27-28° C. as 30-200 ml suspension cultures in 490 or 850 ml roller bottles in Express Five TM SFM medium (Gibco BRL, Cat. # 10486-025), supplemented with L-Glutamine to 16.5 mM. (Gibco BRL, Cat. # 25030). 1×10⁶cells in 2-ml medium were seeded Into 6-well multidishes just before infection. The infected cells were incubated 96 hours at 27-28° C., and samples of 150 μl were taken and prepared for PCR analysis. To the 150 μl were added 350 μl H₂O, 50 μl 10% SDS and DNA was extracted from this mixture by a phenol/chloroform extraction and precipitation by ethanol and finally the DNA pellet was resuspended in 10 μH₂O. 1 μl hereof was used for PCR amplification using primers specific for the human DPPI sequence and conditions similar to the ones used for amplification of the coding regions of DPPI (Example 1 and 2). When the PCR product was analysed on an agarose gel, a band of the expected size was obtained. Samples from cells infected with wild type AcNPV did not show this band. Recombinant viruses were also analysed for their ability to mediate expression of active DPPI. For this purpose, samples of culture medium from the infected High Five TM cells described immediately above were taken 120 hours post infection and tested using the assay as described in Example 7. When isolates were selected after the PCR analysis and the activity analysis, master virus stocks were prepared by a subsequent amplification of the plaque eluates on Sf9 cells in monolayer (Gruenwald et al. (1993) Procedures and Methods Manual, 2nd ed., Pharmigen, San Diego, Calif. p. 52-53). High titre viral stocks (>1×10⁸ plaque forming units/ml used for scaling up the production of prepro-DPPI were obtained by further amplification on 50 ml Sf9 cell cultures in suspension (1×10⁶ cells/ml) using a multiplicity of infection (MOI) of 0.1-0.2. Virus titres were determined by plaque assay.

### Example 5

Expression of Extracellular DPPI in Insect Cell/Baculovirus System (BEVS)

Viral stocks of CLU10-4 and CLU70-1, prepared as described in Example 4, were used to infect suspension cultures of High Five TM cells in roller bottles in Express Five TM SFM medium supplemented with L-Glutamine to 16.5 mM. Infection of insect host cells In different experiments was carried out at a multiplicity of Infection (MOI) of 1-10. Cell densities at the time of Infection were varied in the range of 5×10⁵ to 2×10⁶ cells/ml. Cell culturing was continued for up to 6 days and samples were collected and analysed for DPPI activity on each day from day 2 (48 hours post infection). DPPI enzyme activity was measured in the clarified media (15,000× g, 2 minutes). Recombinant DPPI was secreted as unprocessed proenzyme and the proteolytic maturation required for activity was initiated in the medium. Activation was completed in vitro by 1-2 days of incubation at low pH but for analytical purposes, activation could also be accelerated by papain treatment as described in (Lauritzen et al. (1998) Protein Expr. Purif. 14, 434442). 5 days post infection, recombinant DPPI levels of 0.1-1 unit/ml of culture were achieved with both the human and the rat DPPI. A typical time course of DPPI activity in the culture medium from a 150 ml High Five TM culture seeded to 1×10⁶ cells/ml and infected with CLU70-1 at an MOI of 2 is shown in the table below.

TABLE 1

| | without | with |
|---|---|---|
| | papain activation | papain activation |
| 72 hours post infection (units/ml) | 0.02 | 0.26 |
| 96 hours post infection (units/ml) | 0.09 | 0.40 |
| 120 hours post infection (units/ml) | 0.543 | 0.629 |

### Example 6

Scale-Up of Secreted Human and Rat Pro-DPPI Production

High Five TM cells grown in Express Five TM SFM medium supplemented with L-Glutamine to 16.5 mM were used to produce secreted human prepro-DPPI in 0.3-2.5 litre production scales and rat prepro-DPPI. in 4500 ml production scale. Approximately 1.2×10⁶ cells/ml in volumes of 150 ml per 850 ml roller bottle were infected with a viral stock of CLU70-1 or PCLU10-4 at an MOI of 10. The roller bottles were incubated at 27-28° C. with a speed of 12 rpm. 72 hours post infection, the medium was cleared from cells and cell debris by centrifugation at 9000 rpm, 10° C., 15 minutes.

### Example 7

Measurement of DPPI Activity

DPPI activity was determined by spectrophotometric measurement of the Initial rate of hydrolysis of the chromogenic substrate Gly-Phe-p-nitroanilide (Sigma). One unit was defined, as the amount of enzyme required to convert 1 μmol of substrate per minute at the described conditions. For samples of culture medium, the assay was performed as follows:

1 part of medium was mixed with 2 parts of 200 mM cysteamine in water and 1 part of either water (without papain activation) or 1 mg/ml papain (with papain activation). After 10 min of incubation at 37° C., the mixture was supplemented 1:1 with fresh 200 mM cysteamine. 50 μl of sample was immediately diluted with 950 μl of preheated assay buffer containing substrate (20 mM citric acid, 150 mM NaCl, 1 mM EDTA, 4 mM Gly-Phe-p-nitroanilide, pH 4.5) and the change in absorbance at 405 nm (37° C.) was measured. More concentrated samples of DPPI were diluted an additional 10 times with assay buffer prior to the final mixing with 200 mM cysteamine and assay buffer containing substrate. The background level of hydrolysis of Gly-Phe-p-nitroanilide in the supernatant from wild-type AcNPV Infected High Five cell cultures measured both with and without papain addition corresponded to <0.02 units of DPPI activity per ml culture.

### Example 8

Preparative Scale Purification of Recombinant DPPI and Pro-DPPI

Human and rat DPPI were purified from the insect cell supernatant by ammonium sulphate fractionation followed by hydrophobic interaction and anion exchange chromatography (Lauritzen et al. (1998) Protein Expr. Purif. 14, 434442). To the clarified supernatant from e.g. 1800 ml of rrDPPI or rhDPPI infected cell culture was added (NH₄)₂SO₄ to 2 M and cysteamine-HCl and EDTA to 5 mM. The pH was then adjusted to 4.5 using 1 M citric acid followed by stirring for 20 min. Centrifugation and filtration removed the resulting precipitate. The conditioned supernatant was loaded at a flow-rate of 10-15 ml/min onto a Butyl Sepharose FF (Pharmacia, Uppsala, Sweden) column (5.3 cm²×35 cm) equilibrated with 20 mM citric acid, 2 M (NH₄2SO₄, 100 mM NaCl, 5 mM cysteamine, 5 mM EDTA, pH 4.5. The column was washed with 100 ml equilibration buffer and DPPI was eluted with a linear gradient of 2-0 M (NH₄)₂SO₄ in equilibration buffer over 100 ml (6.6 ml/min). Fractions containing DPPI activity were pooled and incubated at 4° C. for 18-40 hours to obtain a fully processed form. The preparation was then desalted on a Sephadex G25 F (Pharmacia, Uppsala, Sweden) column (5.3 cm²×35 cm) equilibrated with 5 mM sodium phosphate, 1 mM EDTA, 5 mM cysteamine, pH 7.0. This buffer was also used to equilibrate a Q-Sepharose FF (Pharmacia, Uppsala, Sweden) column (2 cm²×10 cm) onto which the collected G25 F eluate was loaded at a flow rate of 3 ml/min. After washing the column, DPPI was step-eluted with desalting buffer containing 250 mM NaCl. Following SDS-PAGE and Western blotting (Lauritzen et al. (1998) Protein Expr. Purif. 14, 434-442) of mature recombinant rat and human DPPI, the following N-terminal amino acid sequences were determined on a Procise 494 from Applied Biosystems:

TABLE 2
Determined N-terminal amino acid sequence:

| Enzyme | Res. pro-part | Heavy chain | Light chain |
|---|---|---|---|
| Human DPPI | DTPAXX- | ILHLPT- | DPFNPF- |
| Rat DPPI | DTPAXXTYPDL- | ILSLPE- (80%) | DPFNPF- |
| | | LSLPES- (15%) | |
| | | LPESWD- (5%) | |

It is clear from the results that mature DPPI produced in the insect cell/baculovirus system was processed very similar to native DPPI (Dolenc et al. (1995) J. Biol. Chem. 270, 21626-21631, Nikawa et al. (1992) Eur. J. Biochem. 204, 381-393).

Human and rat pro-DPPI were expressed using a similar baculovirus constructs and purified by the same overall procedures as applied for the production of mature human and rat DPPI, as described in (Lauritzen et al. (1998) Protein Expr. Purif. 14,434-442). However, to isolate pro-DPPI, cells were harvested two days earlier (on day three) and the purification was performed at pH 7.0. Surprisingly, pro-DPPI did not bind to the Q Sepharose (Pharmacia, Uppsala/SE) column in the final purification step, but was eluted in the flow-through in a substantially pure form. Typically, 10-25 mg of pure pro-DPPI was isolated per litre of culture. Pro-DPPI was concentrated in a dialysis bag covered by a 2-3 cm layer of PEG6000 for 1 hour. The enzyme and proenzyme preparations were finally formulated by addition of 1120 volume of 5 M NaCl and 1.35 volumes of 86-88% glycerol. All chromatographic steps were carried out at 20-25° C. and the formulated product was stored at −20° C.

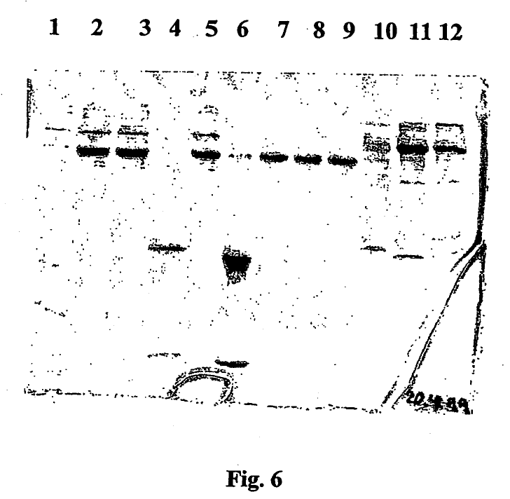

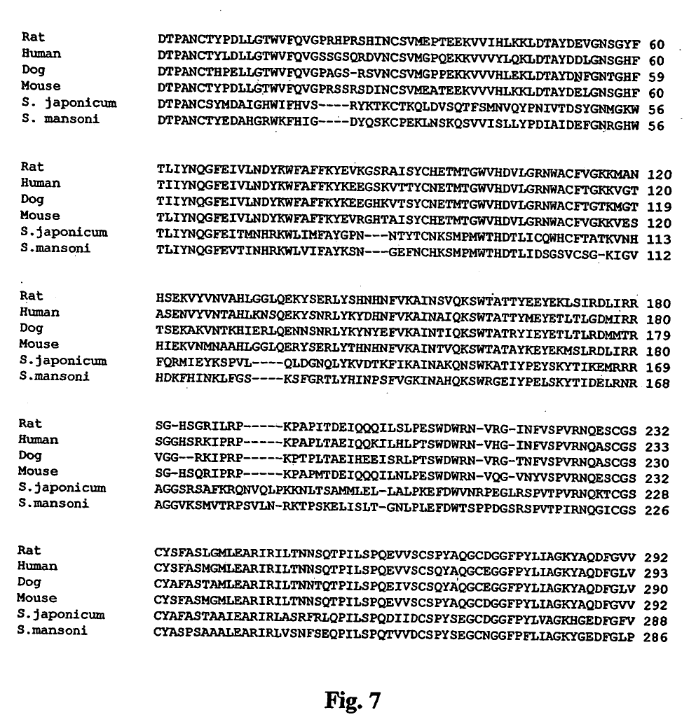

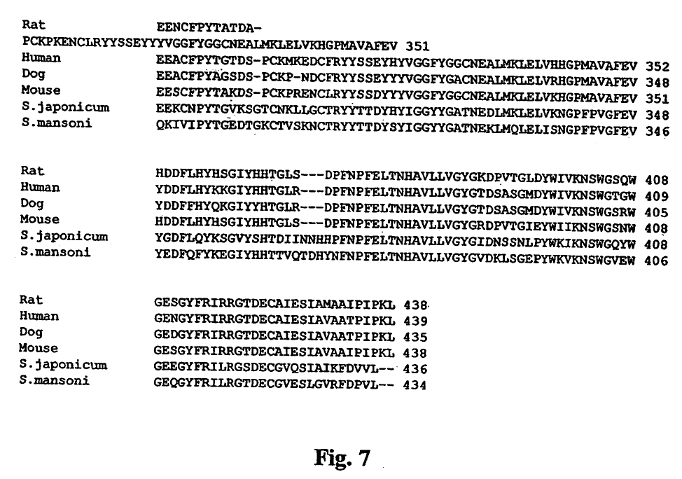

FIG. 6 illustrates the purity and stability of the different fractions (analysed by SDS-PAGE) during the purification of human pro-DPPI. Upon incubation for 18 hour at 4-6° C., pro-DPPI purified on a Butyl-Sepharose column (pane 2) was stable at pH 7.0 (lane 3) but not at pH 4.5 (lane 4). However, surprisingly, pro-DPPI was found in the flow-through from the Q-Sepharose column (lane 7) and was stable both at pH 7.0 (lane 8) and at pH 4.5 (lane 9) upon incubation for 18 hour at 4-6° C. Also, most surprisingly, impurities were found to bind to the Q-Sepharose column and could be eluted by the same concentration of NaCl that Is normally used to elute active DPPI (lane 10, 11 and 12).

### Example 9

Analysis of N-gycosylation

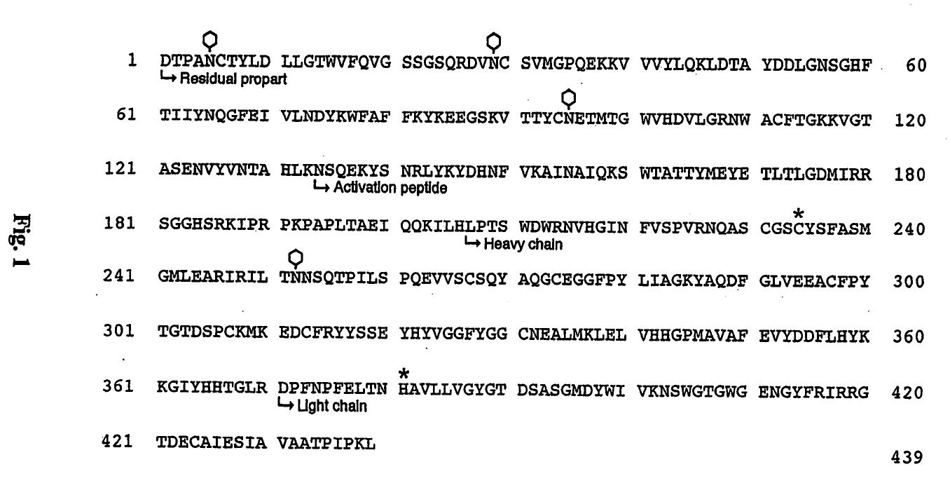

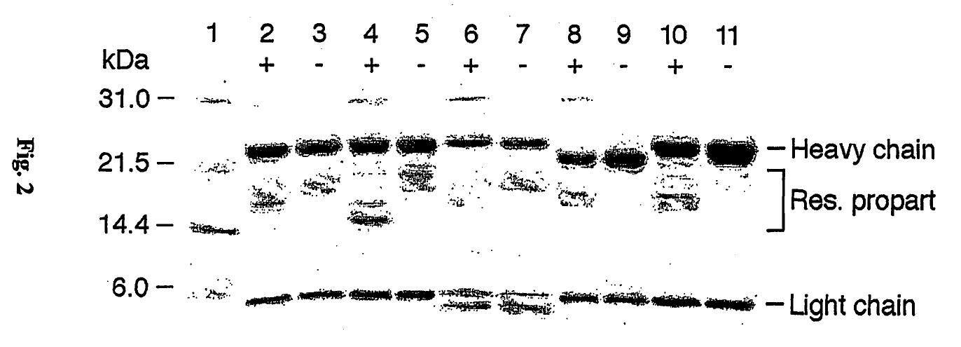

The level of N-glycosylation on the recombinant human enzyme was investigated by individually destroying each of the four potential N-glycosylation sites by substituting the required Ser or Thr residue In the consensus sequence for N-glycosylation with an Ala residue (-N-X-S/T- to N-X-A-). The results of SDS-PAGE analysis of the purified mutants showed that all four potential sites (N5, N29, N95 in the propeptide and N252 in the heavy chain) were glycosylated (FIG. 2) as in the native enzyme (Cigic et al. (1998) Biochim. Biophys. Acta, 1382, 143 150). Compared to wild type DPPI (lanes 10 and 11), the N5 (lanes 2 and 3) and N29 (Vanes 4 and 5) glycosylation site mutants showed increased residual pro-part and unchanged heavy chain mobilities. The apparent molecular mass of both residual pro-parts were further reduced by endoglycosidase H (Invitrogen, Carlsbad, Calif.) treatment (all samples in lanes with even numbers were treated) according to the manufacturers recommendations. Similarly, deletion of the third site at N95 (lanes 6 and 7) affected the migration and homogeneity of the residual pro-part However, no further mass reduction was observed following endoglycosidase H treatment (lane 6 vs. lane 7). Finally, elimination of the site at N252 (lanes 8 and 9) resulted in an increased heavy chain mobility. The residual pro-parts of this mutant and of the wild type DPPI displayed the same migration pattern following endoglycosidase H treatment (lane 8 vs. lane 9). Molecular mass markers are shown in lane 1. The modified asparagine residues are marked with hexagons in FIG. 1.

### Example 10

Analyses of the Oligomenc Structure of Pro-DPPI by Gel Filtration Chromatography

The oligomeric structures of recombinant human DPPI and pro-DPPI were determined by gel filtraion analysis on an analytical Superdex 200 column (Pharmacia, Uppsala, Sweden) in 20 mM sodium phosphate buffer pH 7.0, 500 mM NaCl and at a flow rate of 0.7 ml/min (22° C.). Catalase, aldolase, His-tagged pyroglutamyl peptidase I, bovine serum albumin, ovalbumin and lysozyme were used as standards. The proenzyme migrated with an elution volume corresponding to a mass of 77 kDa whereas mature DPPI migrated with a mass of 140 kDa. The results suggest that the recombinant human proenzyme is a dimer and that the mature form is a tetramer.

### Example 11

Structural Analyses by Circular Dichroism Spectroscopy

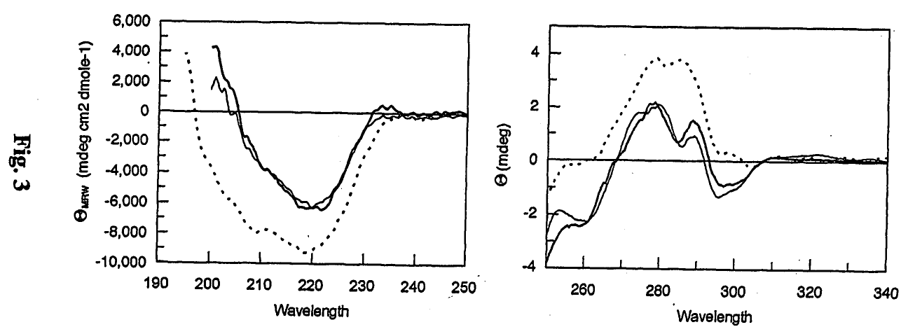

Circular dichroism was measured at 25° C. with an AVIV 62A DS circular dichroism spectrometer (Lakewood, N.J.). Cells with pathlengths of 1 cm (near-UV region, 320-250 nm) or 0.1 cm (far-UV region, 250-200 nm) were used. Prior to measurements, the enzymes were dialysed against 50-mM sodium phosphate buffer, pH 6.0, containing 0.5 mM EDTA. Both the far-(left panel) and near-(right panel) UV CD spectra of native and recombinant human DPPI (FIG. 3) were very similar confirming that the native and recombinant forms have the same content of secondary structural elements and that the aromatic residues are located in very similar environments. However, the spectra of an unfunctional Cys234 to Ser234 mutant of pro-DPPI differed substantially from those of the mature proteins, reflecting the contribution of the activation peptide.

### Example 12

Identification of Human Cathepsins L and S as Potential Activators of Human DPPI In Vivo

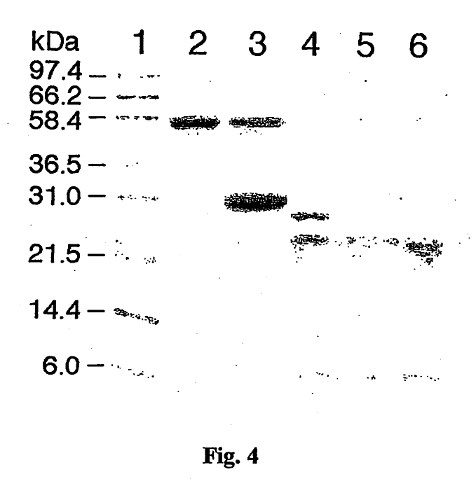

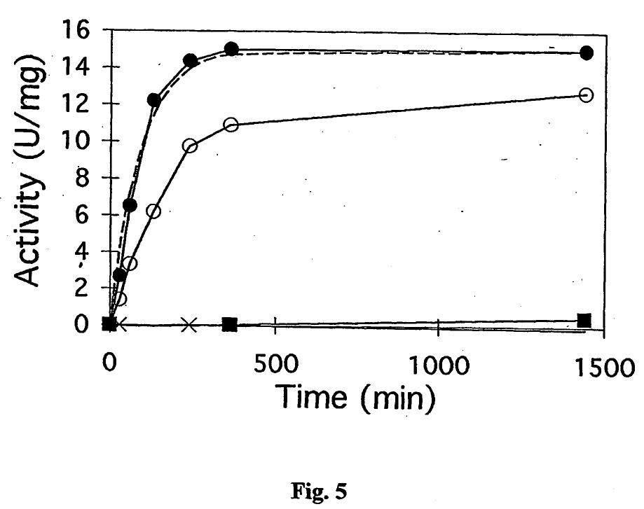

Pure pro-DPPI (5 μM) was treated with E64 titrated recombinant human cathepsins B (5 μM), S(1 μM) and L (0.2 μM) in 20 mM citric acid, 150 mM NaCl, 1 mM EDTA, 10 mM DTT, pH 4.5 or pH 4.5-6.9 (cathepsin B only) (FIGS. 4 and 5). Cathepsins L (FIG. 4: lane 5, FIG. 5: filled circles) and S (lane 4, open circles) processed wild type recombinant human pro-DPPI into active human DPPI. The activation of pro-DPPI by cathepsin L followed pseudo-first order kinetics (broken line). Papain processed wild type recombinant rat pro-DPPI into active rat-DPPI and could increase the specific activity of recombinant human pro-DPPI about 2,000-fold. By contrast, cathepsin B showed little or no processing of human recombinant pro-DPPI at pH 4.5 (lane 3, filled squares), pH 5.3, 6.1 and 6.9 (data not shown). Pro-DPPI Incubated without addition of endopeptidase (lane 2, crosses) or In presence of active recombinant human DPPI (results not shown) was not processed.

### Example 13

Cloning of Prepro-DPPI cDNA from Other Organisms

Nucleotide sequences encoding prepro-DPPI or pro-DPPI from species other than rat or human can also be cloned for recombinant expression of the proteins. For example, murine cDNA from e.g. kidney is prepared by conventional methods e.g. as described in (Lauritzen et al. (1998) Protein Expr. Purif. 14, 434-442). The published nucleotide sequences encoding murine DPPI (McGuire et al. (1997) Biochim. Biophys. Acta. 1351, 267-273) is used as a basis for designing oligonucleotide primers to specifically amplify the coding region by PCR from the cDNA pool. The primers must be designed such as to contain restriction sites in addition to homologous sequences. The product is then cloned using the restriction sites into a suitable transfer or expression vector. For example, the products can be inserted into pCLU10-4 (described in Example 1) to create a baculovirus transfer vector. Proper vector construction must of course be confirmed by nucleotide sequencing of the coding region on the constructed plasmid before using the plasmid for co-transfection of insect cells and preparation of virus stocks.

The nucleotide sequences encoding DPPI proteins from species with unknown sequence can be cloned by various methods known in the art, for example by PCR cloning. The sequences known to be well-conserved among DPPI proteins from different species and amino acid sequences obtained with e.g. digestive fragments of the proteins purified from natural sources is used as basis for designing polynucleotide primers. These primers are then used to amplify fragments of the encoding sequences from cDNA pools. Through a number of steps including DNA sequencing of PCR products, new primer design and new PCR amplification steps, the entire coding sequence is isolated and cloned into e.g. a baculovirus transfer vector.

### Example 14

Expression of Prepro-DPPI in Pichia pastoris

Nucleic acids containing sequences coding for pro-DPPI proteins are amplified by PCR with primers containing restriction sites, in addition to homologous sequences. The products are then cloned using the restriction sites into a Pichia pastoris expression vector, e.g. pPICZalfa (Invitrogen). By cloning into pPICZalfa, the pro-DPPI coding sequence can be fused to the sequence for the alfa-factor secretion signal. A direct fusion between the alfa-factor C-terminal residues ( . . . LeuGluLysArg) and the pro-DPPI N-terminal residues (AspThrPro . . . ) can be made by cloning into the XhoI restriction site in the 3′ end of the alfa-factor sequence. The 5′ end PCR primer should then contain a XhoI site and In addition a short sequence replacing the deleted part of the alfa-factor sequence. Any of the downstream restriction sites in the vector-cloning site can be used. for ligation of the 3′ end of the pro-DPPI sequence. Recombinant vectors are transformed into an E. coli host strain for propagation of plasmid, and proper vector construction is confirmed by nucleotide sequencing of the cloned region.

Preparations of selected recombinant vectors are linearised and transformed by electroporation into a compatible host like the Pichia pastoris strains KM71 or GS115.

Transformed clones are identified by Zeocin resistance and PCR screening.

Transformed Pichia clones are used for expression experiments using methanol to induce the production of the recombinant product controlled by the alcohol oxidase promoter AOX1. At time points post-induction (for example 0, 6, 12, 24, 36, 48, 60, 72, 84 and 96 hours) samples are taken from the cultures and the level of pro-DPPI and processed and active DPPI analysed e.g. by DPPI activity assay and Coomassie-stained SDS-PAGE and western blot. Positive clones are identified by e.g. western blot on the culture supernatants. Conditions resulting in the highest levels of pro-DPPI are hereby determined. Expression is scaled up, and the resulting culture supernatants are cleared by centrifugation. The expressed pro-DPPI protein is isolated, yielding a purified pro-DPPI protein.

### Example 15

Detection of Pro-DPPI as a Marker Using Anti-Pro-DPPI Antibodies

To quantify pro-DPPI in e.g. blood, serum, faeces, urine, cerebro or cerebra-spinal fluid or tissue extract by indirect enzyme linked immunosorbent assay (ELISA), apply 100 μl of sample diluted in a coating solution such as 50 mM sodium carbonate (pH 9.6), 20 mM Tris-HCl (pH 8.5) or 10 mM PBS (pH 7.2) to a suitable container, preferably a microtitre plate well. The final concentration of pro-DPPI is preferably 0.1-50 μg/ml. Incubate 1 hour at room temperature. Empty plate and tap out residual liquid. To minimise non-speceic binding to the microtitre plate well surface, add 300 μl blocking solution containing e.g. 0.1-1.0 μg/ml of BSA, non-fat dry milk, casein or gelatin to each well. Incubate 5 minutes, empty plate and tap out residual liquid. Add 100 μl diluted anti-pro-DPPI antibody in 1× blocking solution to each well and incubate 1 hour at room temperature. The optimum concentration of antibody depends on the specificity and binding affinity of the antibody. Empty plate, tap out residual liquid. Unbound primary antibody is removed by washing 3 to 5 times with wash solution, typically 0.1 M phosphate-buffered saline or Tris-buffered saline (pH 7.4) with a detergent such as Tween 20 (0.01%-0.1% v/v). After washing, 100 μl diluted horseradish peroxidase conjugated secondary antibody in 1× blocking solution is added. Incubate 1 hour at room temperature and wash 35 times as before. Remove wash solution. To quantify the amount of pro-DPPI, dispense 100 μl tetramethyl benzidin substrate in 30% hydrogen peroxide into the well. The development of colour can be quantified at 450 nm after addition of an appropriate stop solution (e.g. 1 M sulfuric add) or monitored using a plate reader.

The detection of pro-DPPI can also be performed by use of secondary antibodies labelled with other enzymatic or non-enzymatic markers and by similar methods, e.g. direct ELISA, sandwich ELISA or RIA, known to people skilled in the art of immunochemistry.

Refer nces

Cigic, B., Krizaj, I., Kralj, B., Turk, V., and Pain, R. H. (1998) Stoichiometry and heterogeneity of the pro-region chain in tetrameric human cathepsin C. Biochim. Biophys. Acta 1382, 143-150.

Clackson, T., Hoogenboom, H. R., Griffths, A. D., and Winter, G. (1991) Nature 352, 624-628.

Demirov, D., Sarafian, V., Kremensky, I., and Ganev, V. (1999) Evidence for protein splicing in the endoplasmic reticulum-Golgi intermediate compartment Biochim. Biophys. Acta 1448, 507-511.

Dolenc, I., Turk, B., Pungercic, G., Ritonja, A., and Turk, V. (1995) Oligomeric structure and substrate induced inhibition of human cathepsin C. J. Biol. Chem. 270, 21626-21631.

Gruenwald, S., and Heitz, J. (1993) Baculovirus Expression Vector System: Procedures and Methods Manual, 2nd ed., Pharmigen, San Diego, Calif.

Hart, T. C., Hart, P. S., Bowden, D. W., Michalec, M. D., Callison, S. A., Walker, S. J., Zhang, Y., and Firatli, E. (1999) Mutations of the cathepsin C gene are responsible for Papillon-Lefèvre syndrome. J. Med. Genet. 36, 881-887.

Ishidoh, K., Muno, D., Sato, N., and Kominami, E. (1991) Molecular cloning of cDNA for rat cathepsin C. Cathepsin C, a cysteine proteinase with an extremely long propeptide. J. Biol. Chem. 266, 16312-16317.

Jolliffe, L. K. (1993) Humanized antibodies: enhancing therapeutic utility through antibody engineering. Int. Rev. Immunol. 10, 241-250.

Kohler, G, and Milstein, C. (1975) Continuous cultures of fused cells secreting antibody of predefined specificity. Nature 256, 495-497.

Lauritzen, C., Pedersen, J., Madsen, M. T., Justesen, J., Martensen, P. M., and Dahl, S. W. (1998) Active recombinant rat dipeptidyl aminopeptidase I (cathepsin C) produced using the baculovirus expression system. Protein Expr. Purif. 14, 434-442.

Mainferme, F., Wattiaux, R., and von Figura, K. (1985) Synthesis, transport and processing of cathepsin C in Morris hepatoma 7777 cells and rat hepatocytes. Eur. J. Biochem. 153,211-216.

Marks, J. D., Hoogenboom, H. R., Bonnert, T. P., McCafferty, J., Griffith's, A. D., and Winter G. (1991) By-passing immunization. Human antibodies from V-gene libraries displayed on phage. J. Mol. Biol. 222,581-597.

McGuire, M. J., Lipsky, P. E., and Thiele, D. L. (1992) Purification and characterization of dipeptidyl peptidase I from human spleen. Arch. Biochem. Biophys. 295,280-288.

Muno, D., Ishidoh, K, Ueno, T., and Kominami, E. (1993) Processing and transport of the precursor of cathepsin C during its transfer into lysosomes. Arch. Biochem. Biophys. 306, 103-110.

Nikawa, T., Towatari, T., and Katunuma, N. (1992) Purification and characterization of cathepsin J from rat liver. Eur. J. Biochem. 204, 381-393.

Paris, A., Strukelj, B., Pungercar, J., Renko, M., Dolenc, I., and Turk, V. (1995) Molecular cloning and sequence analysis of human preprocathepsin C. FEBS Lett. 369,326-330.

Presta, L. G. (1992) Antibody engineering. Curr. Opin. Biotechnol. 3, 394-398.

Ronald, A., Stimson, W. H. (1998) The evolution of immunoassay technology. Parasitology 117, Suppl. 13-27.

Self, C. H., and Cook, D. B. (1996) Advances in immunoassay technology. Curr. Opin. Biotechnol. 7, 60-65.

Thompson, J. D., Higgins, D. G., and Gibson, T. J.(1994) CLUSTAL W: improving the sensitivity of progressive multiple sequence alignment through sequence weighting, position-specific gap penalties and weight matrix choice. Nucleic Acids Res. 22, 4673-4680.

Toomes, C., James, J., Wood, A. J., Wu, C. L., McCormick, D., Lench, N., Hewitt, C., Moynihan, L., Roberts, E., Woods, C. G., Markham, A., Wong, M., Widmer, R., Ghaffar, K. A., Pemberton, M., Hussein, I. R., Temtamy, S. A., Davies, R., Read, A. P., Sloan, P., Dixon, M. J., and Thakker, N. S. (1999) Loss-of-function mutations in the cathepsin C gene result in periodontal disease and palmoplantar keratosis. Nat Genet. 23, 421-424.

1. An isolated, substantially pure polypeptide comprising a single chain polypeptide consisting of about 390-470 amino acid residues, said single chain polypeptide being at least 40% identical to a proenzyme of human dipeptdyl peptidase I (pro-DPPI), as shown in SEQ ID No. 1.

2. An isolated, substantially pure polypeptide according to claim 1, comprising an internal activation peptide sequence that is at least 25% identical to an internal activation peptide of human pro-DPPI, as shown in SEQ ID No. 1 ranging from amino acid No. 134 to amino acid No. 206.

3. An isolated, substantially pure polypeptide, according to claim 1 or 2 that is at least 70%-100% identical to human pro-DPPI, as show in SEQ ID No. 1.

4. An isolated polypeptide that corresponds to human pro-DPPI, as shown in SEQ ID No. 1.

5. A method for isolating a substantially pure polypeptide, characterised by constructing a nucleic acid vector encoding a DPPI proenzyme N-terminally fused to a signal sequence for isolating a substantially pure polypeptide, according to any of claims 1-4.

6. A method for isolating a substantially pure polypeptide, according to claim 5, wherein the nucleic acid vector encoding a DPPI proenzyme is fused N-terminally to a signal sequence of DPPI preproenzyme.

7. A method for Isolating a substantially pure polypeptide, according to claim 6, wherein the signal sequence is a rat signal of DDPI preproenzyme.

8. A method according to claim 5-7, characterised by the polypeptide being isolated from a DPPI producing host under conditions that inhibit the processing of pro-DPPI.

9. A method according to any of the claims 5-8, the method comprising:
a) inserting a nucleic acid fragment containing a nucleic acid sequence encoding a polypeptide according to any of claims 1-4 into a vector encoding said nucleic acid N-terminally fused to the signal sequence,
b) transfecting or infecting or transforming a suitable host with said vector produced in step a),
c) cultivating or growing said host under suitable conditions for expressing the polypeptide, and
d) harvesting and purifying the polypeptide under conditions that inhibit the processing of the proenzyme.

10. A method according to any of claims 5-9, wherein the method is further characterised by the introduction of conditions that stabilise the proenzyme.

11. A method according to any of claims 5-10, wherein at least one proteolytic enzyme capable of modifying the proenzyme and/or converting the proenzyme into an active enzyme is inhibited, blocked, deleted and/or made unfunctional.

12. A method according to any of claims 5-11, wherein said host is a cell that is adapted for expression of recombinant proteins such as a prokaryotic, lower eukaryotic, mammalian or insect cell.

13. A method according to any of claims 5-12, characterised by yielding an outcome of 1-500 mg of substantially pure protein purified per litre of culture and/or per 1-8×10⁹ cells.

14. A method according to any of claims 5-13, wherein the vector is a viral vector, such as a baculovirus vector selected from the group consisting of Autographica californica multiple nuclear polyhedrosis virus and Bombyx mori nuclear polyhedrosis virus.

15. A method according to any of claims 5-14, wherein said polypeptide is purified from the host at the latest about 4 days, preferably about 3 days, after the transfection.

16. A method according to any of claims 5-15, wherein said substantially purified polypeptide corresponds to a recombinant proenzyme of human DPPI.

17. A method according to any of claims 5-16, wherein a polypeptide according to claims 1-4 is selectively isolated from a mixture of polypeptides containing both the processed and unprocessed form of said polypeptide, said method being characterised by employing
a) ammonium sulphate fractioning,
b) hydrophobic interaction,
c) desalting, and
d) anion exchange chromatography.

18. A substantially pure polypeptide obtainable by a method according to any of claims 5-17.

19. A substantially pure polypeptide obtained by a method according to any of claims 5-17.

20. Use of a substantially pure polypeptide according to claims 1-4, 18 or 19 for the identification of natural pro-DPPI binding proteins, characterised by reacting pro-DPPI with pure or impure preparations of one or more potential pro-DPPI binding proteins and investigating for complex formation employing methods such as non-denaturing gel electrophoresis or gel filtration chromatography.

21. An immunologic composition comprising a substantially pure polypeptide as defined in any of claims 1-4, 18 or 19.

22. An immunologic composition according to claim 21, which further comprises an immunologically and pharmaceutically acceptable carrier, vehicle and/or adjuvant.

23. Use of a substantially pure polypeptide as defined In any of claims 1-4, 18 or 19 for the production of pro-DPPI antibodies.

24. A method for Inhibiting the processing of pro-DPPI, characterised by administering a substance that binds to pro-DPPI and thereby inhibits the processing of pro-DPPI into mature DPPI, thereby inhibiting the localised catalytic activity of DPPI.

25. Use of an inhibitory substance according to claim 24 as a medicament.

26. Use of a substantially pure polypeptide as defined in any of claims 1-4, 18 or 19 as a medicament intended for therapeutic or diagnostic application to conditions and/or diseases comprising asthma, psoriasis, inflammatory bowel disease, graft-versus-host-disease, cardiac hypertrophy, heart failure, atherosclerorosis, perionditis, rheumatoid arthritis, allergic rhinitis, arteriosclerosis, organ allograft rejection, multiple sclerosis and myasthenia grais and conditions and/or diseases related to excessive or-reduced apoptosis in a mammal.

27. Use of a monoclonal antibody according to any of claims 24-25 for treating conditions and/or diseases comprising asthma, psoriasis, inflammatory bowel disease, graft-versus-host-disease, cardiac hypertrophy, heart failure, atherosderorosis, perionditis, rheumatoid arthritis, allergic rhinitis, arteriosclerosis, organ allograft rejection, multiple sclerosis and myasthenia grais and conditions and/or diseases related to excessive or reduced apoptosis in a mammal, by inhibiting the processing of pro-DPPI, and thereby inhibiting the localised catalytic activity of DPPI.

28. Use of a polypeptide according to claim 14, 18 or 19 for manufacturing a medicament for treating conditions and/or diseases comprising asthma, psoriasis, inflammatory bowel disease, graft-versus-host-disease, cardiac hypertrophy, heart failure, atherosclerorosis, perionditis, rheumatoid arthritis, allergic rhinitis, arteriosclerosis, organ allograft rejection, multiple sclerosis and myasthenia grais and conditions and/or diseases related to excessive or reduced apoptosis in a mammal,

29. A method for the manufacturing of a medicament intended for therapeutic or diagnostic application to conditions and/or diseases comprising asthma, psoriasis, inflammatory bowel disease, graft-versus-host-disease, cardiac hypertrophy, heart failure, atherosclerorosis, perionditis, rheumatoid arthritis, allergic rhinitis, arteriosclerosis, organ allograft rejection, multiple sclerosis and myasthenia grais and conditions and/or diseases related to excessive or reduced apoptosis in a mammal, characterised in that a substantially pure polypeptide as defined in any of claims 1-4, 18 or 19 is used.

30. A method for the manufacturing of a medicament intended for therapeutic or diagnostic application in conditions and/or diseases comprising asthma, psoriasis, inflammatory bowel disease, graft-versus-host-disease, cardiac hypertrophy, heart failure, atherosclerorosis, perionditis, rheumatoid arthritis, allergic rhinitis, arteriosclerosis, organ allograft rejection, multiple sclerosis and myasthenia grais and conditions and/or diseases related to excessive or reduced apoptosis in a mammal, characterised in that a polyclonal or a monoclonal antibody according to any of claims 24-25 is used.

31. A method according to claim 29 or 30 wherein the mammal is a human.


SEQUENCE LISTING (1 sequence)

SEQ ID NO: 1 - protein, 439 aa, Homo sapiens
DTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNTAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEVVSCSQYAQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL